(19) United States
(12) Patent Application Publication
(10) Pub. No.: US 2012/0207766 A1
(43) Pub. Date: Aug. 16, 2012

(54) INTRACELLULAR TOLL-LIKE RECEPTORS PATHWAYS AND AXONAL DEGENERATION

(76) Inventors: Athena Soulika, Sacramento, CA (US); David Pleasure, Sacramento, CA (US)

(21) Appl. No.: 13/361,018

(22) Filed: Jan. 30, 2012

Related U.S. Application Data
(63) Continuation of application No. PCT/US2010/042892, filed on Jul. 22, 2010.
(60) Provisional application No. 61/228,145, filed on Jul. 23, 2009.

(51) Int. Cl. A61K 39/395; A61K 31/47; A61K 31/517; A61K 31/55; A61K 31/44; A61K 31/7088; A61K 31/551; A61P 33/10; A61P 33/02; A61P 31/12; A61P 25/00; A61P 37/06; A61K 38/17; A61K 31/135

(52) U.S. Cl. 424/172.1; 514/17.7; 514/311; 514/266.1; 514/217; 514/656; 514/324; 514/214.02; 514/44 A

(57) ABSTRACT

The present invention is directed to the identification of novel mechanisms involved in the initiation and spreading of axonal degeneration. In particular, the instant invention relates to the identification of a relationship between Toll-Like Receptors (TLRs) and axonal degeneration and the use of TLR inhibitors as antagonists of axonal degeneration.

### CROSS REFERENCE TO RELATED APPLICATIONS

This application is a continuation of International Patent Application No. PCT/US2010/042892, filed Jul. 22, 2010, which claims priority to U.S. Provisional Application Ser. No. 61/228,145, filed Jul. 23, 2009, the contents of which are incorporated herein in its entirety.

### FIELD OF THE INVENTION

The present invention is directed to the identification of novel mechanisms involved in the initiation and spreading of axonal degeneration. In particular, the instant invention relates to the identification of a relationship between Toll-Like Receptors (TLRs) and axonal degeneration and the use of TLR inhibitors as antagonists of axonal degeneration.

### BACKGROUND OF THE INVENTION

Acute Transverse Myelitis (ATM) is an inflammatory disease of the central nervous system (CNS) that affects both adults and children and often leads to irreversible neurological deficits. It is histologically characterized by CNS perivascular mononuclear infiltrates, demyelination and axonal damage. The main reason for the onset and persistence of neurological deficits is the accumulation of degenerating axons. Evidence from patients and animal models support the theory that early interventions that would limit the “spreading” of axonal injury would also lead to decreased clinical symptoms and an improved prognosis.

The events leading in concert to the onset of neurological deficits, due to spinal cord inflammation, have been the focus of investigation for many years. Recently, it was established that axonal degeneration is the “prodrome” of possibly permanent neurological symptoms. Shriver & Dittel, Am J Pathol 169, 999-1011 (2006); Coleman et al., Nat Rev Neurosci 6, 889-98 (2005); and Papadopoulos et al., Exp Neurol 197, 37385 (2006).

Studies in children suffering from acute transverse myelitis (ATM) show that there is a rapid onset of severe symptoms lasting up to a year. After that, neurological deficits usually subside or completely resolve, but in 30-40% of the cases residual chronic disability remains. Miyazawa et al., Pediatr Int 45, 512-6 (2003); Paine & Byers, AMA Am T Dis Child 85, 151-63 (1953); and Dunne et al., Dev Med Child Neurol 28, 198-204 (1986).

Anti-inflammatory treatment reduces pain during the peak of the disease, in some cases, but does not change the long term prognosis. Studies in children with ATM are rare. Much of the knowledge about inflammatory neurological diseases comes from multiple sclerosis; MRI along with histological studies provide strong evidence that the increased degree of disability is due to cumulative axonal degeneration, suggesting that once a certain pathological state is crossed, axons and neurons will deteriorate and will ultimately undergo apoptosis. Bjartmar & Trapp, Neurotox Res 5, 157-64 (2003). These observations underline the importance of neuroprotective, in conjunction with anti-inflammatory, treatments in order to battle inflammation-induced axonal degeneration.

In injured axons vesicular transport is interrupted, causing them to swell. Although axonal swelling was thought to be characteristic of irreversibly damaged axons, studies show that it can also be detected in intact axons or before demyelination and that control of the early stages axonal injury can prevent its further spreading. Shriver & Dittel, Am J Pathol 169, 999-1011 (2006); Stokin et al., Science 307, 1282-8 (2005); Sasaki, S., Acta Neuropathol 110, 48-56 (2005); and Lappe-Siefke et al., Nat Genet. 33, 366-74 (2003). Interventions in the early stages of neurological symptoms that will delay or inhibit the spreading of injury in axons will be instrumental in alleviating permanent and severe forms of disability.

For example, in inflammatory neurological disorders, axonal injury is induced by peripheral immune cells that enter the CNS. CNS-intrinsic innate immune mechanisms also contribute to the onset and severity of neurological deficits in animal models of spinal cord inflammation. Toll-like receptors (TLRs) have received significant attention due to the fact that inactivation of the TLR-devoted signaling molecule, MyD88, in the CNS, leads to decreased disease severity and tissue injury in inflamed spinal cord. Prinz et al., J Clin Invest 116, 456-64 (2006). However it was not until the studies outlined in the instant application that a connection between TLR activity and axonal degeneration was specifically identified.

TLRs can be divided in two groups: plasma membrane-associated (TLR1, 2, 4, 5, 6, 10 (only in human) and -11 (only mouse)) and endolysosomal or intracellular (TLR3, 7, 8 and 9). TLR12 and TLR13 are newly identified mouse receptors; their cellular distribution has not been clarified, but evidence suggests that TLR13 is endolysosomally located. Members of the TLR family are involved in CNS development and processes such as apoptosis and autophagy, but their main function is to participate in innate immune responses: they recognize patterns expressed in non-self and initiate the transcription of inflammatory cytokines mainly through the signaling molecule MyD88 (except TLR3) and transcription factors such as NFκB and AP1. Larsen et al., Sci STKE 2007, pe47 (2007); Salaun et al., Eur J Immunol 37, 3311-8 (2007); Russell & Yates, Nat Immunol 8, 217; 217-8 (2007); and Akira & Takeda, Nat Rev Immunol 4, 499-511 (2004). mRNA copies of most TLRs are upregulated in the CNS during inflammation as shown by RT-PCR analysis. Prinz et al., J Clin Invest 116, 456-64 (2006); and Soulika et al., Journal of Neuroscience Submitted (2009).

Activity of intracellular TLRs is dependent on their translocation to endosomal/lysosomal compartments; the acidic environment in the endolysosomes is necessary for the activity of intracellular TLRs. Nishiya et al., J Biol Chem 280, 37107-17 (2005); Gibbard et al., J Biol Chem 281, 27503-11 (2006); and Ranjith-Kumar et al., J Biol Chem 282, 7668-78 (2007). Translocation occurs only after association with the endoplasmic reticulum protein UNC93B which happens after TLR-ligand exposure. Tabeta et al., Nat Immunol 7, 156-64 (2006); Kim et al., Nature 452, 234-8 (2008); Brinkmann et al., J Cell Biol 177, 265-75 (2007); Latz et al., Nat Immunol 5, 190-8 (2004); and Park et al., Nat Immunol 9, 1407-14 (2008). Mutations of UNC93B in both human and mice result in inhibition of intracellular TLR functions. Brinkmann et al., J Cell Biol 177, 265-75 (2007); and Zhang et al., Immunol Rev 220, 225-36 (2007).

Intracellular TLRs have been implicated in the recognition of viral RNA and DNA and imidazoquinoline derived compounds. Akira et al., Cell 124, 783-801 (2006). Interestingly, TLR3 and 8 were the only TLRs shown to be involved in CNS development; this process was not dependent exclusively on the canonical TLR signaling pathway through MyD88 and NFκB. Ma et al., J Cell Biol 175, 209-15 (2006); and Cameron et al., J Neurosci 27, 13033-41 (2007). This was expected for TLR3, which normally does not utilize MyD88, but surprising for TLR8. On the contrary, another set of studies imply that TLR8 can activate the canonical TLR pathway. Intracerebral administration of TLR8 activators in neonatal mice induced the production of pro-inflammatory cytokines (which requires MyD88 and NFκB). Also, culture of murine or human TLR8-overexpressing cell lines with the TLR8 activators induced MyD88 and NFκB activation. Gorden et al., J Immunol 177, 8164-70 (2006).

Neuronal or axonal expression of other intracellular TLRs (7 and 9) has not been studied. However, TLR9 deletion in the CNS ameliorates the severity of neurological symptoms while TLR3 activity seems to be protective in inflammatory neurological disorders. Prinz et al., J Clin Invest 116, 456-64 (2006); and Touil et al., J Immunol 177, 7505-9 (2006). To date, no studies have addressed TLR7 or TLR8 function in such disorders.

In light of the foregoing, there remains a need in the art to design specific therapeutics to prevent the spreading of axonal injury. By establishing that intracellular TLR signaling contributes to the degeneration of axons, the instant invention identifies new compositions and methods for the prevention of axonal degeneration. Compositions and methods taking advantage of this relationship will be beneficial for patients suffering from neurological deficits induced by myelitis and other axonal degeneration-related diseases.

### SUMMARY OF THE INVENTION

The present invention is based, in part, on the discovery that a new marker for axons displaying signs of injury is an intracellular Toll-Like Receptor (TLR). Intracellular TLRs are innate immune receptors that localize in the lysosomes and participate in innate immune responses. Specifically, intracellular TLRs participate in the initiation and spreading of axonal degeneration in myelitis and related diseases.

In certain embodiments, the present invention is directed to methods of antagonizing axonal degeneration using a TLR inhibitor. In certain embodiments the TLR inhibitor is a small molecule inhibitor, such as, but not limited to, tricyclic TLR inhibitors, substituted quinoline compounds and substituted quinazoline compounds. In certain embodiments the tricyclic TLR inhibitor is selected from the group comprising mianserin, desipramine, cyclobenzaprine, imiprimine, ketotifen, and amitriptyline. In certain embodiments the TLR inhibitor is a biologic, such as, but not limited to, an anti-TLR-related antigen antibody, an inhibitory peptide, or an inhibitory nucleic acid.

In certain embodiments, the present invention is directed to pharmacologic TLR inhibitors that are employed to antagonize axonal degeneration. In alternative embodiments, the present invention is directed to genetic inhibition of intracellular TLRs, which results in antagonism of axonal degeneration.

In certain embodiments the present invention is directed to methods for treating axonal loss associated with myelitis comprising inhibiting the activity of a Toll-Like Receptor to inhibit axonal degeneration, thereby treating axonal loss associated with myelitis.

In certain embodiments the present invention is directed to methods for treating axonal loss associated with multiple sclerosis comprising inhibiting the activity of a Toll-Like Receptor to inhibit axonal degeneration, thereby treating multiple sclerosis.

In certain embodiments the present invention is directed to methods for treating axonal loss associated with systemic lupus erythematosis comprising inhibiting the activity of a Toll-Like Receptor to inhibit axonal degeneration, thereby treating axonal loss associated with systemic lupus erythematosis.

In certain embodiments the present invention is directed to methods for treating axonal loss associated with acute corticospinal tract trauma comprising inhibiting the activity of a Toll-Like Receptor to inhibit axonal degeneration, thereby treating axonal loss associated with acute corticospinal tract trauma.

In certain embodiments the present invention is directed to methods for treating axonal loss associated with viral infection comprising inhibiting the activity of a Toll-Like Receptor to inhibit axonal degeneration, thereby treating axonal loss associated with viral infection.

### BRIEF DESCRIPTION OF THE FIGURES

FIG. 1 depicts acute focal axonal injury post-MOG peptide within focal inflammatory infiltrates. A ventrolateral lumbar spinal cord inflammatory focus is shown on day 14 post-MOG peptide, stained (from left to right) for the axonal damage marker amyloid precursor protein (APP), the neutrophil marker Ly6G, nuclei with DAPI, and the merged images.

FIG. 2 depicts SMI32+ damaged axons, some surrounded by MBP+ myelin (arrow), others demyelinated (arrowhead) within a spinal cord inflammatory focus on day 12 post-MOG peptide.

FIG. 3 depicts symmetric loss of EYFP+CST axons in the L5 dorsal columns of MOG peptide-myelitis mice. Differences in axon numbers between controls (n=4), day 35 post-MOG peptide (n=6), and day 101 post-MOG peptide (n=4) mice were statistically significant (p<0.02, ANOVA). Values are means±SD. Dorsal CST axon counts in day 21 post-MOG peptide mice did not differ significantly from those in day 21 post-CFA control mice

FIG. 4 depicts high power views of L5 CSTs of day 101 post-CFA control mice (left panels) and day 101 post-MOG peptide mice (right panels), immunostained with SMI312, showing marked axonal loss in the MOG peptide myelitis mice

FIG. 5 depicts dorsal CSTs of day 35 post-CFA control (upper panels) and day 35 post-MOG peptide myelitis Emx-Cre/Thy1-STOP-EYFP mice (lower panels) are shown. Note the accumulations of CD68+ and IBA1+ microglia/macrophages in the myelitic specimen, which also showed depletion of EYFP+CST axons.

FIG. 6 depicts L5 spinal cord dorsal CSTs of Emx-Cre/Thy1-STOP-EYFP 35 day CFA control (left panel), 35 day MOG peptide (center panel) and 101 MOG peptide mice (right panel), immunostained for GFAP. Dorsal CST astrogliosis was more prominent on day 35 post-immunization than 2 months later.

FIG. 7 depicts SMI32+ axons from MOG peptide mice contained immunoreactive TLR8. TLR8 immunoreactivity was not detected in axons of CFA- or normal-control mice (not shown).

FIG. 8 depicts colocalization of TLR8 with the retrograde endosomal marker protein, RAB7, in axons of MOG peptide myelitis mice.

FIG. 9 depicts data obtained from Unc93b1 bone marrow chimera studies. Data are daily means (+SEMs) of 4 wildtype (WT)→SWT (control) vs 3 WT→UNC mutant (test) bone marrow chimeras. SEM bars are within the circles at late time-points in the control mice.

FIG. 10 depicts dorsal CSTs (located in most ventral fasciculus gracilis) in lumbar cord of a WT→WT control chimeric mouse (left panel) and a WT→UNC mutant (test) chimeric mouse (right panel), 42 days post-MOG peptide. SMI312+ axons are in red, nuclear DAPI in blue. Size bar=20 μm.

FIG. 11 depicts lumbar dorsal funiculi of WT→WT (left 2 panels) and WT→UNC mutant (right 2 panels) mice 42 days post-MOG peptide. Note lesser myelin basic protein (MBP) immunostaining in controls, but similar numbers of IBA+ macrophages in control and test mice. Size bar=100 μm.

FIG. 12 deptics a study indicating that systemic administration of mianserin does not affect MOG-specific Th17 cell generation in the periphery (panel A, each bar represents a single mouse) but diminishes neurological symptoms associated with MOG-myelitis (Panels Band C, n=8).

### DETAILED DESCRIPTION OF THE INVENTION

The present invention is directed to the identification of novel mechanisms involved in the initiation and spreading of axonal degeneration. In particular, the instant invention relates to the identification of a relationship between TLRs and axonal degeneration and the use of TLR inhibitors as antagonists of axonal degeneration.

Unless otherwise defined, all technical and scientific terms used herein have the same meanings as commonly understood by one of ordinary skill in the art to which this invention belongs. Although methods and materials similar or equivalent to those described herein can be used in the practice or testing of the present invention, suitable methods and materials are described below.

Methods of Use

The present invention establishes that axonal intracellular TLR function is implicated in the initiation and spreading of axonal degeneration and that inhibition of TLR function results in antagonism of axonal degeneration. Accordingly, inactivation of axonal intracellular TLRs via the compositions and methods described herein can be used to reduce axonal damage and, in certain instances, decrease the severity of neurological symptoms associated with axonal degeneration.

In certain embodiments the present invention is directed to methods for treating axonal loss associated with myelitis comprising inhibiting the activity of a Toll-Like Receptor to inhibit axonal degeneration, thereby treating axonal loss associated with myelitis.

In certain embodiments the present invention is directed to methods for treating axonal loss associated with multiple sclerosis comprising inhibiting the activity of a Toll-Like Receptor to inhibit axonal degeneration, thereby treating multiple sclerosis.

In certain embodiments the present invention is directed to methods for treating axonal loss associated with systemic lupus erythematosis comprising inhibiting the activity of a Toll-Like Receptor to inhibit axonal degeneration, thereby treating axonal loss associated with systemic lupus erythematosis.

In certain embodiments the present invention is directed to methods for treating axonal loss associated with acute corticospinal tract trauma comprising inhibiting the activity of a Toll-Like Receptor to inhibit axonal degeneration, thereby treating axonal loss associated with acute corticospinal tract trauma.

In certain embodiments the present invention is directed to methods for treating axonal loss associated with viral infection comprising inhibiting the activity of a Toll-Like Receptor to inhibit axonal degeneration, thereby treating axonal loss associated with viral infection.

Toll Like Receptor Inhibitors

In certain embodiments, the present invention is directed to specific TLR inhibitors capable of antagonizing axonal degeneration. In certain, non-limiting, embodiments, the TLR inhibitor of the present invention is a small molecule TLR inhibitor or a biologic TLR inhibitor. In certain embodiments more than one TLR inhibitor can be used in combination with one or more other TLR inhibitor or one or more other therapeutic agent.

In certain embodiments, the TLR inhibitor of the instant invention is a small molecule inhibitor of TLR activity. Exemplary small molecule inhibitors include, but are not limited to, mianserin, desipramine, cyclobenzaprine, imiprimine, ketotifen, and amitriptyline. Alternative small molecule inhibitors of TLR activity include, but are not limited to, other known tricyclic TLR inhibitors, as well as the substituted quinoline and quinazoline compounds published patent application US 2005/0119273.

In certain embodiments, the present invention is directed to a biologic inhibitor of TLR activity. Exemplary biologic TLR inhibitors include, but are not limited to, TLR-inhibiting antibodies, TLR-inhibiting peptides, and TLR-inhibiting nucleic acids.

In certain embodiments the present invention is directed to a TLR-inhibiting antibody. The term “antibody” as used in this section refers to an intact antibody or an antigen binding fragment thereof. The antibodies of the present disclosure can be generated by a variety of techniques, including immunization of an animal with the TLR-related antigen of interest followed by conventional monoclonal antibody methodologies e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes. The TLR-related antigen may be all or a portion of a TLR protein sequence, in the case of inhibitory antibodies that bind the TLR directly, or an antigen may be all or a portion of an alternative protein, the binding of which results in the inhibition of TLR activity.

One preferred animal system for preparing hybridomas is the murine system. Hybridoma production is a very well-established procedure. Immunization protocols and techniques for isolation of immunized splenocytes for fusion are known in the art. Fusion partners (e.g., murine myeloma cells) and fusion procedures are also known.

An antibody preferably can be a human, a chimeric, or a humanized antibody. Chimeric or humanized antibodies of the present disclosure can be prepared based on the sequence of a non-human monoclonal antibody prepared as described above. DNA encoding the heavy and light chain immunoglobulins can be obtained from the non-human hybridoma of interest and engineered to contain non-murine (e.g., human) immunoglobulin sequences using standard molecular biology techniques. For example, to create a chimeric antibody, murine variable regions can be linked to human constant regions using methods known in the art (see e.g., U.S. Pat. No. 4,816,567 to Cabilly et al.). To create a humanized antibody, murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et In one non-limiting embodiment, the antibodies of this disclosure are human monoclonal antibodies. Such human monoclonal antibodies directed against TLR-related antigens can be generated using transgenic or transchromosomic mice carrying parts of the human immune system rather than the mouse system. Such transgenic and transchromosomic mice include mice referred to herein as the HuMAb Mouse® (Medarex, Inc.), KM Mouse® (Medarex, Inc.), and XenoMouse® (Amgen). Additional methods for obtaining fully human antibodies to TLR-related antigens are described in Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum Antibod Hybridomas 3:81-85; Huse et al. (1989) Science 246:1275-1281; McCafferty et al., Nature (1990) 348:552-554; Griffiths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Hoogenboom et al. (1991) Nuc Acid Res 19:4133-4137; and Barbas et al. (1991) PNAS 88:7978-7982.

In certain embodiments, the present invention is directed to inhibitory nucleic acids capable of decreasing TLR gene expression or TLR protein activity. Such inhibitory nucleic acids include, but are not limited to, antisense nucleic acids, ribozymes, and siRNA nucleic acids. In certain embodiments, the inhibitory nucleic acids of the present invention functions by inhibiting either transcription or translation of a particular target gene.

In certain embodiments, the inhibitory nucleic acid of the present invention is an antisense nucleic acid molecule, i.e., a molecule that is complementary to the coding strand of a TLR-encoding nucleic acid. An antisense oligonucleotide can be, for example, but not by way of limitation, about 5, 10, 15, 20, 25, 30, 35, 40, 45, or 50 or more nucleotides in length. An antisense nucleic acid of the invention can be constructed using chemical synthesis or by via enzymatic synthesis reactions using procedures known in the art. For example, an antisense nucleic acid (e.g., an antisense oligonucleotide) can be chemically synthesized using naturally occurring nucleotides or modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

In certain embodiments the inhibitory nucleic acid is a ribozyme. Ribozymes are catalytic RNA molecules that exhibit ribonuclease activity, and which are capable of cleaving a single-stranded nucleic acid, such as an mRNA, to which they have a complementary region. Thus, ribozymes (e.g., hammerhead ribozymes as described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding an TLR protein of interest can be designed based upon the nucleotide sequence of that TLR protein. For example, and not by way of limitation, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742).

In certain embodiments, the inhibitory nucleic acid is a siRNA. siRNA-mediated transcript “knockdown,” is a technique which has emerged as a standard way of specifically and potently inhibiting the expression of large numbers of genes. The siRNA of the invention can comprise partially purified RNA, substantially pure RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribonucleotides.

In certain embodiments, one or both strands of the siRNA of the invention can also comprise a 3′ overhang. As used herein, a “3′ overhang” refers to at least one unpaired nucleotide extending from the 3′-end of a duplexed RNA strand. Thus in one embodiment, the siRNA of the invention comprises at least one 3′ overhang of from 1 to about 6 nucleotides (which includes ribonucleotides or deoxyribonucleotides) in length, preferably from 1 to about 5 nucleotides in length, more preferably from 1 to about 4 nucleotides in length, and particularly preferably from about 2 to about 4 nucleotides in length.

The siRNA of the invention can be targeted to any stretch of approximately 19-25 contiguous nucleotides in any of the target mRNA sequences. Techniques for selecting target sequences for siRNA are given, for example, in Tuschl T et al., “The siRNA User Guide,” revised Oct. 11, 2002, the entire disclosure of which is herein incorporated by reference. Generally, a target sequence on the target mRNA can be selected from a given cDNA sequence corresponding to the target mRNA, preferably beginning 50 to 100 nt downstream (i.e., in the 3′ direction) from the start codon. The target sequence can, however, be located in the 5′ or 3′ untranslated regions, or in the region nearby the start codon.

Pharmaceutical Compositions

In certain embodiments the TLR inhibitor of the present invention is prepared in the form of a pharmaceutical composition. Such pharmaceutical compositions can be provided in any of a variety of formations. For example, but not by way of limitation, the pharmaceutical composition of the present invention may be employed in such forms, both sterile and non-sterile, such as capsules, liquid solutions, liquid drops, emulsions, suspensions, elixirs, creams, suppositories, gels, soft capsules, sprays, inhalants, aerosols, powders, tablets, coated tablets, lozenges, microcapsules, suppositories, dragees, syrups, slurries, granules, enemas or pills. Any inert carrier can be used, such as saline, or phosphate buffered saline, stabilizers, propellants, encased in gelatin capsule or in a microcapsule or vector that aids administration or any such carrier in which the compositions used in the method of the present invention have suitable solubility properties for use in the methods of the present invention.

In a further embodiment the subject composition can be delivered alone or in conjunction with a dispersion system. In some embodiments the dispersion system is selected from the group consisting of, but not limited to macromolecular complexes, nanocapsules, microspheres, beads and lipid based systems. Lipid-based systems optionally include oil-in-water emulsions, micelles, mixed micelles, or liposomes.

In certain embodiments a subject composition is in the form of a pharmaceutically acceptable solution, which may routinely contain pharmaceutically acceptable concentrations of salt, buffering agents, preservatives, compatible carriers, adjuvants and optionally other therapeutic ingredients. Such composition can contain additives for example: disintegrants, binders, coating agents, swelling agents, lubricants, flavorings, sweeteners or solubilizers and the like. In certain embodiments a subject immunogenic composition is administered in its neat form or in the form of a pharmaceutically acceptable salt. In certain embodiments, the subject composition is freeze-dried (lyophilized) for long term stability and storage in a solid form. The freeze-dried method is known to those skilled in the art.

The present invention may be illustrated by the use of the following non-limiting examples

### EXAMPLES

### Example 1

### Induction of MOG Peptide Myelitis

Induction of MOG peptide myelitis, and clinical evaluation. MOG peptide myelitis is elicited in 3 month old male mice by subcutaneous flank administration of 300 μg of rodent MOG peptide 35-55 emulsified in CFA. The mice receive 75 ng of pertussis toxin intraperitoneally on days 0 and 2, and are weighed and scored daily for neurological deficits by 2 blinded observers. Neurological scoring is on a 5 point scale: limp tail or waddling gait=1; limp tail and waddling gait=2; single limb paresis and ataxia 2.5; double limb paresis=3; single limb paralysis and paresis of a second limb=3.5; full paralysis of 2 limbs=4; and death (or sacrifice owing to inanition=5) (Bannerman et al, Brain 128:1877-1886 2005; Bannerman et al., Glia 55:57-64 2008; and Soulika et al, J Neurosci 29:14965-14979 2009).

Histological evaluation. Mice are anesthetized with ketamine/xylazine, then perfused with phosphate-buffered saline (PBS) followed by 4% paraformaldehyde in PBS. With the assistance of NIH Image J software, axons in the dorsal CSTs in spinal cord L5 and C2 cross-sections are counted, using EYFP for labeling when the mice carry EmxCre and Thy1-STOP-YFP transgenes (Bareyre et al, Nature Med 11:1355-1360 2005; Soulika et al, J Neurosci 29:14965-14979 2009), and SM1312 neurofilament immunostaining (Frischer et al, Brain 132:1175-1189 2009; and Soulika et al, J Neurosci 29:14965-14979 2009). Non-biased stereology (Guo et al, J Neurosci 29:7256-7270 2009) is used to quantify corticospinal projection neurons in motor sensory cortical layer 5.

Sample size, and statistical analysis. Groups of 6 test mice and 6 in each control group is sufficient for statistical analysis. The experiments are long-term, and occasional mice do not survive, so each study begins with 8 mice in each group. Because the scoring of clinical deficits is non-parametric, clinical data is examined for significant differences between groups by Kruskal-Wallis one-way analysis of variance. CST axon and corticospinal neuron counts is examined for significant differences between groups by one-way ANOVA.

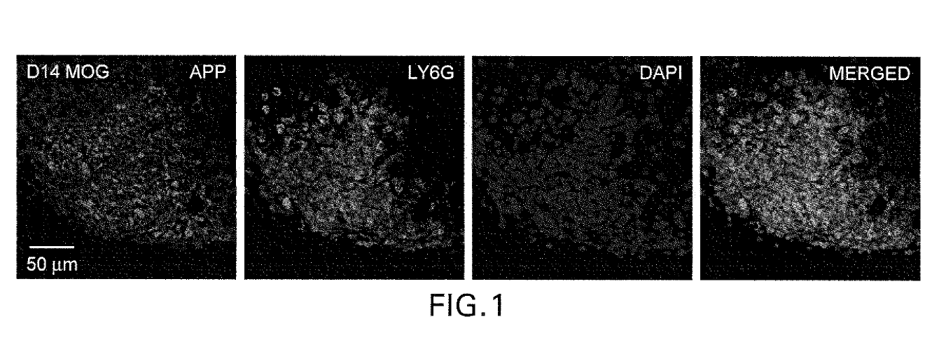

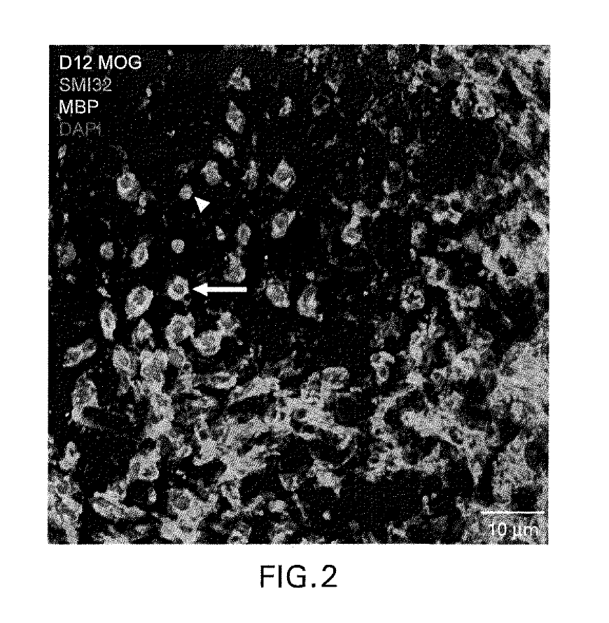

Acute multifocal axonal lesions in MOG peptide myelitis. Damage to axons within early inflammatory lesions is illustrated in FIG. 1. Acute axonal damage is also demonstrable using the SMI32 monoclonal antibody, which recognizes hypophosphorylated NF—H epitopes expressed in motor neuron perikarya, but not normally present in white matter (Bannerman et al, Brain 128:1877-1886 2005) (FIG. 2).

Progressive, symmetrical loss of CST axons in MOG peptide myelitis. Virtually all small diameter (0.5-1.2 mm) murine CST axons, which constitute more than 95% of total CST axons, are in the dorsal CSTs, situated in the ventral-most portions of the dorsal fasciculi. Larger diameter axons at the margins of the dorsal CSTs are principally ascending dorsal root ganglion axons (Ren et al, Brain Res Bull 31:13-22 1993).

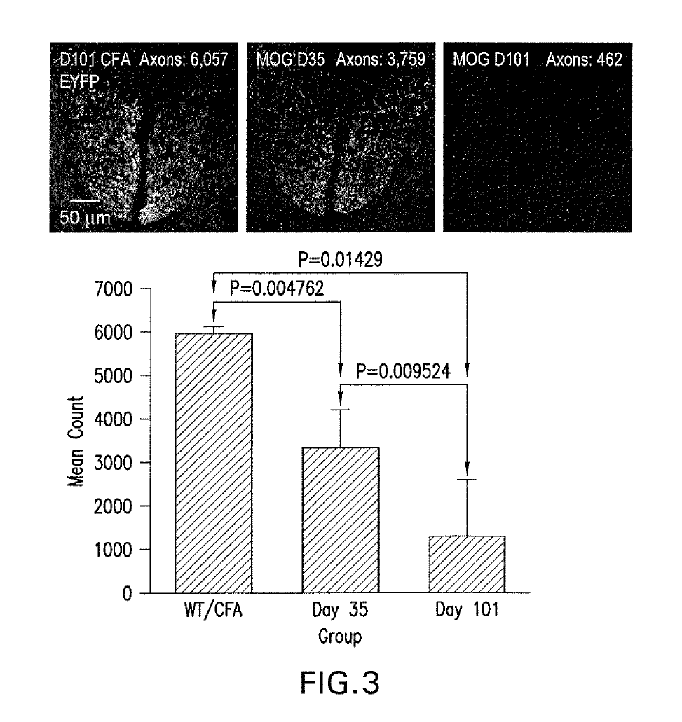

Using a genetic labeling technique employing Emx-Cre/Thy1-STOP-EYFP transgenic mice to discriminate CST axons in the spinal cord dorsal funiculi (Gorsky et al, J Neurosci 22:6309-6314 2002; Bareyre et al, Nature Med 11:1355-1360 2005), it is confirmed that Emx-Cre-driven neuronal recombination in motor-sensory cortex, judged by EYFP labeling, is virtually 100%, and progressive symmetrical loss of small diameter CST axons in MOG peptide myelitis is demonstrated (FIG. 3).

Comparing numbers of axons in the right vs left dorsal CSTs of individual Emx-Cre/Thy1-STOP-EYFP mice yielded regression coefficients of 0.974 at day 35 post-MOG peptide, and 0.956 at day 101 post-MOG peptide (Snedecor and Cochran, Statstical methods, Ed 8. Ames, Iowa: Blackwell 1989). Thus, the CST axonal loss associated with MOG peptide myelitis is delayed in onset, and bilaterally symmetrical, results which are more compatible with a neurodegenerative than an acute multifocal inflammatory process.

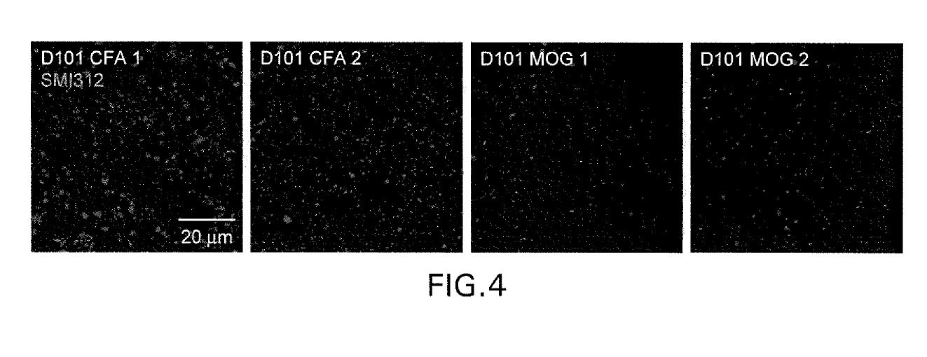

To confirm this progressive diminution in numbers of CST axons, spinal cord cross-sections are immunostained with SMI312, which recognizes multiple phosphorylated neurofilament epitopes (Frischer et al, Brain 132:1175-1189 2009), which demonstrated a profound loss of small axons in the dorsal CSTs of the MOG peptide myelitis mice (FIG. 4).

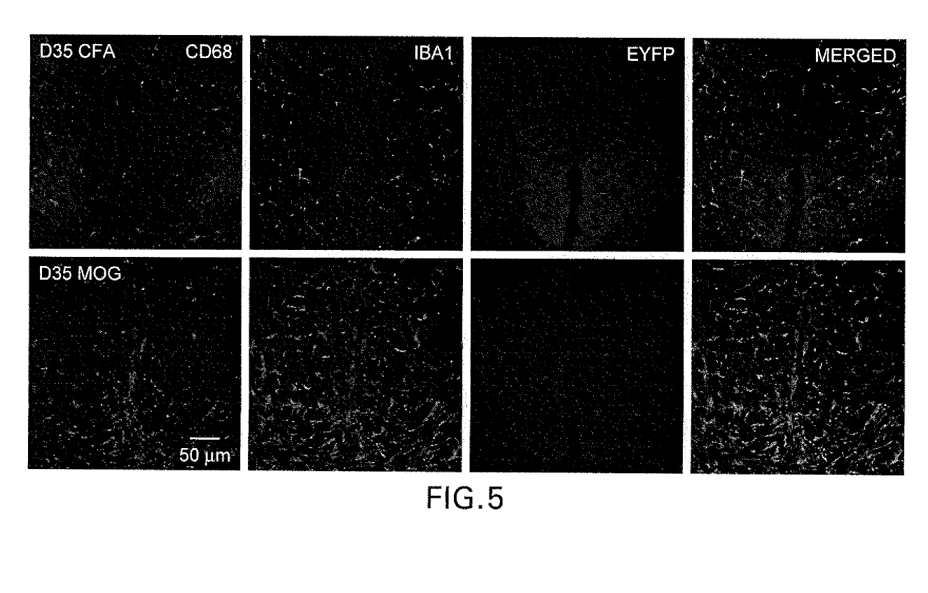

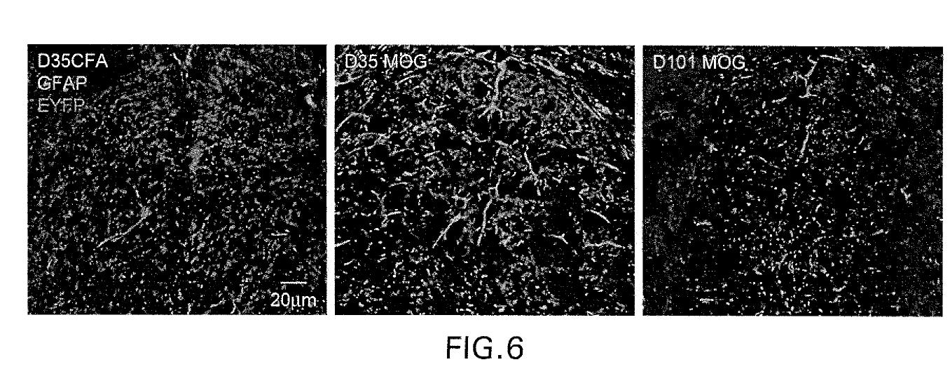

Morphological evidences of innate immune activation in dorsal CSTs of MOG peptide myelitis mice. By day 35 post-MOG peptide, the dorsal CSTs of mice immunized with MOG peptide have become infiltrated by Ibal+ and CD68+ macrophages (FIG. 5), and contain hypertrophic astroglia (FIG. 6).

Cortical neurons are lost in MOG peptide myelitis. Though TUNEL+(Gavrieli et al, J Cell Biol 119:493-501 1992) and activated caspase-3+ neurons are not observed in motor cortex in the myelitic mice, stereological analysis of day 105 post-MOG peptide mice vs day 105 post-CFA control mice show a 37% reduction in numbers of CTIP2+ corticospinal projection neurons (Arlotta et al, Neuron 46:207-221 2005; Tuoc et al, J Neurosci 29:8335-8349 2009) in the MOO peptide myelitis mice (n=5 in each group, p<0.05, unpaired T test).

CNS immune milieu in myelitis. Flow cytometry and qRT/PCR for immune cell type-specific mRNAs show accumulations of CD4+ and CD8+ lymphocytes, neutrophils, and CD11b+ monocyte-derived macrophages in CNS between days 14 and 21 post-MOG peptide, with their gradual clearance from CNS over ensuing months. Levels of CD8 mRNA fall slower than other immune cell-associated mRNAs (Soulika et al, J Neurosci 29:14965-14979 2009). To appraise CNS innate immunity in MOG peptide myelitis mice, spinal cord mRNAs encoding 140 proteins involved in innate immunity are assayed by qRT/PCR (Soulika et al, J Neurosci 29:14965-14979 2009). Selected results for TLRs and TLR signaling proteins are shown in Table 1. Note that all of these transcripts become substantially elevated by day 14 post-MOG peptide immunization, when the mice first became symptomatic, and that most remain elevated more than 3 months post-immunization.

TABLE 1
TLR and TLR-associated mRNAs:

| | day 7 | | day 10 | | day 14 | | day 21 | | day 35 | | day 98 | |
|---|---|---|---|---|---|---|---|---|---|---|---|---|
| | CFA | MOG | CFA | MOG | CFA | MOG | CFA | MOG | CFA | MOG | CFA | MOG |
| TLR1 | 1.5 | 2.4 | 1.8 | 2.0 | 1.3 | 15.7 | 1.2 | 16.3 | 1.8 | 19.7 | 0.8 | 3.5 |
| TLR2 | 0.7 | 1.8 | 0.9 | 1.5 | 0.8 | 18.1 | 0.6 | 8.4 | 0.6 | 5.8 | 0.9 | 5.7 |
| TLR3 | 1.0 | 1.3 | 1.3 | 1.1 | 1.4 | 2.4 | 1.4 | 2.9 | 0.8 | 1.2 | 1.1 | 1.4 |
| TLR4 | 1.2 | 1.6 | 1.0 | 1.5 | 1.2 | 8.1 | 1.2 | 5.4 | 1.7 | 10.0 | 1.1 | 2.2 |
| TLR6 | 0.7 | 1.1 | 0.7 | 0.8 | 0.9 | 4.1 | 0.4 | 4.4 | 1.3 | 7.3 | 0.7 | 1.3 |
| TLR7 | 1.1 | 1.1 | 1.2 | 1.1 | 0.7 | 7.6 | 1.0 | 9.4 | 0.9 | 5.9 | 0.5 | 1.6 |
| TLR8 | 1.8 | 2.7 | 1.7 | 2.0 | 1.2 | 20.5 | 1.3 | 13.0 | 2.3 | 19.2 | 1.0 | 3.8 |
| TLR9 | 1.1 | 1.4 | 1.2 | 1.0 | 1.1 | 5.0 | 1.1 | 5.2 | 2.0 | 11.4 | 1.0 | 2.0 |
| TLR11 | 1.1 | 1.5 | 2.1 | 2.0 | 1.9 | 25.6 | 1.2 | 21.8 | 1.7 | 15.0 | 1.2 | 7.8 |
| TLR12 | 1.6 | 1.3 | 1.6 | 1.3 | 1.1 | 19.5 | 1.3 | 15.3 | 1.2 | 6.4 | 1.5 | 5.0 |
| TLR13 | 1.7 | 1.5 | 2.0 | 1.3 | 1.0 | 22.2 | 0.8 | 9.7 | 1.0 | 7.7 | 1.1 | 4.2 |
| MyD88 | 0.9 | 1.3 | 1.5 | 1.5 | 1.3 | 4.4 | 1.2 | 3.6 | 1.8 | 10.5 | 1.3 | 1.8 |
| Ticam2 | 1.1 | 1.6 | 1.3 | 1.2 | 0.9 | 11.7 | 1.3 | 11.2 | 1.9 | 16.1 | 1.2 | 3.1 |

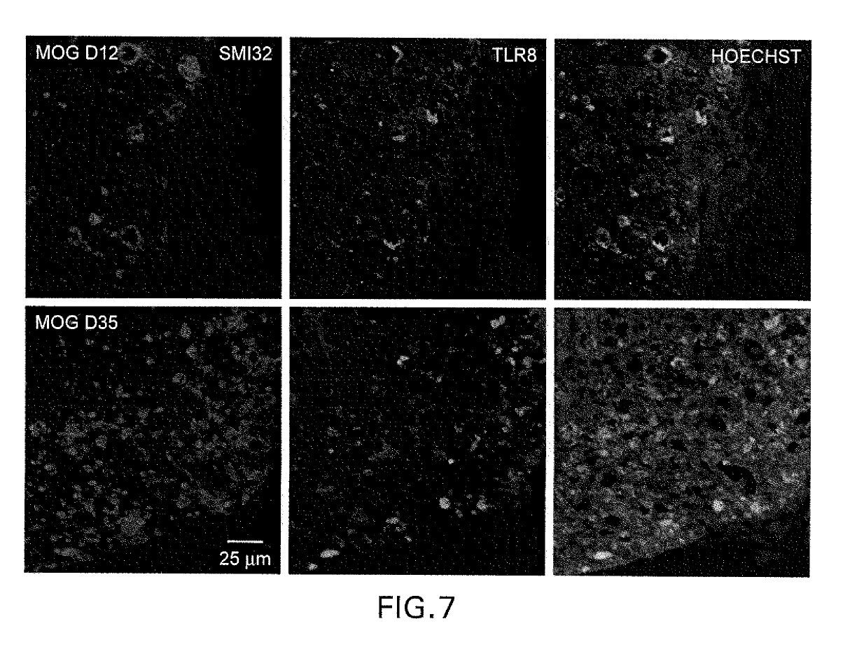

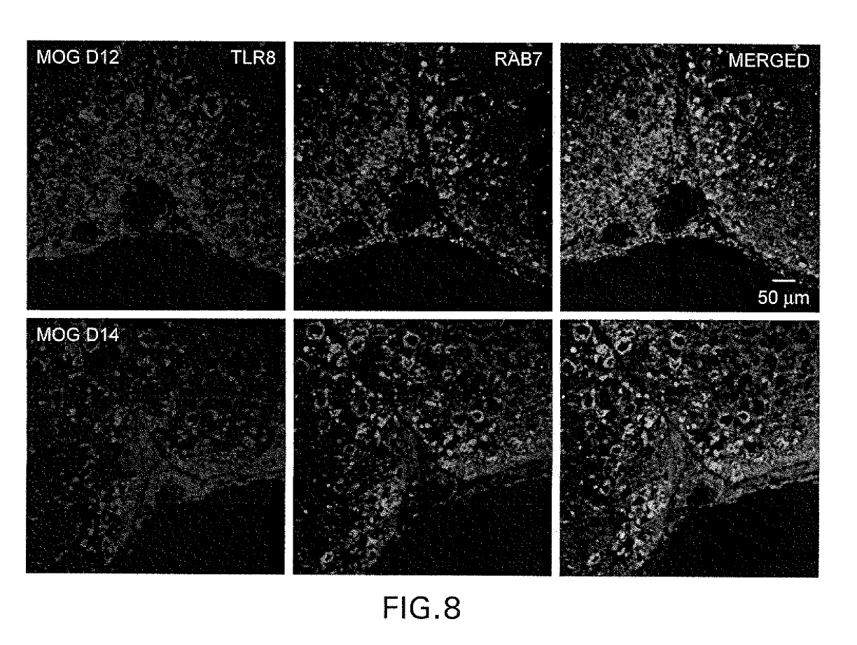

TLR8 accumulated in SMI32+ axons (FIG. 7), where it was often co-localized with RAB7, a protein associated with retrograde-transported endosomes. (FIG. 8).

### Example 2

### Unc93b1 Bone Marrow Chimera Studies

The case that progressive CST axon loss in this spinal cord inflammation model is a consequence of persistently elevated CNS TLR signaling can be supported if there is substantially less dorsal CST axon loss in bone marrow chimeric mice lacking functional CNS Unc93b1. To demonstrate this approach, 3 peripheral wild-type Unc93b1/CNS mutant Unc93b1 (test) and 4 peripheral wild-type Unc93b1/CNS wild-type Unc93b1 (control) bone marrow chimeric mice were produced. Axons in the dorsal CSTs of these mice were visualized by SMI312 (pan-neurofilament) immunostaining.

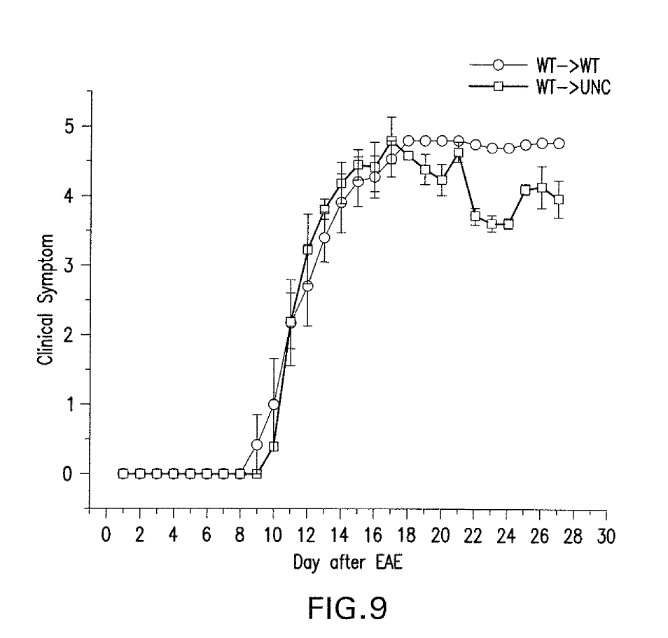

Clinical scores in the test and control chimeric mice through day 35 post-MUG peptide immunization are shown in FIG. 9. The severity of neurological deficits is slightly diminished during the early chronic phase of myelitis in mice in which CNS endolysosomal signaling is suppressed by the Unc93b1 mutation.

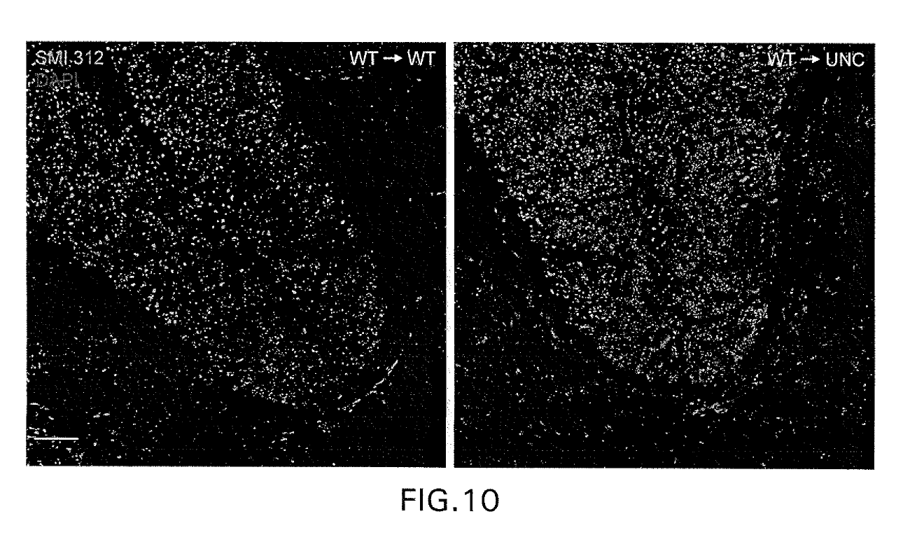

These chimeric mice were then sacrificed. A lumbar spinal cord cross-sectional view of the dorsal CSTs of a representative mouse from each of the two groups, immunostained for axons with SMI312, is shown in FIG. 10. Dorsal CST axons were more numerous in those chimeric mice that expressed inactive mutant Unc93b1 in CNS than in those that expressed wild-type Unc93b1 in CNS.

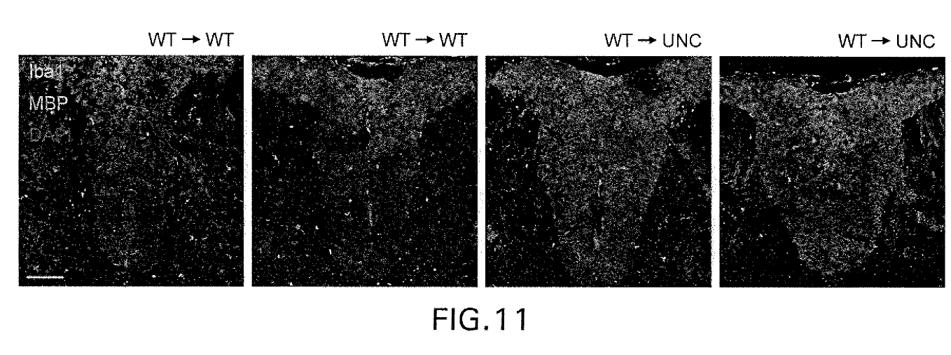

Myelin was also better preserved in the dorsal funiculi of the irradiation bone marrow chimeric mice that lacked functional CNS Unc3b1, but numbers of IBA1+ macrophages were similar in the two groups of mice (FIG. 11).

### Example 3

### Ablating CNS Endolysosomal TLR Signaling Prevents Progressive Post-Myelitic CST Axon Loss

The endolysosomal TLRs, some of which signal by non-canonical pathways, must be transported to endolysosomes by an Unc93b1-mediated process prior to their activation by oligonucleotide ligands. Using irradiation bone marrow chimeric mice that express only an inactive allele of Unc93b1 in CNS, it is possible to establish that ablating CNS endolysosomal TLR signaling prevents progressive post-myelitic CST axon loss.

The endolysosomal TLRs (TLRs 3, 7, 8, 9 and 13) are induced in CNS inflammatory disorders, and persistent elevations in their mRNAs in MOO peptide myelitis have been documented (Bsibsi et al, J Neuropathol Exp Neurol 61:1013-1021 2002; Soulika et al, J Neurosci 29:14965-14979 2009). Two endolysosomal TLRs (TLR3 and, in the mouse, neuronal TLR8) signal via non-MyD88-dependent pathways, the others via both canonical and non-canonical pathways (Ma et al, J Cell Biol 175:209-215 2006). However, signaling by each of this group of TLRs commences only after their transport to endolysosomes, which takes place by an Unc93b1-dependent process (Tabeta et al, Nature Immunol 7:156-164 2006; Brinkmann et al, 2007; Conley, Trends Immunol 28:99-101 2007; Kim et al, Nature 452:234-239 2008; Saitoh and Miyake, Immunol Reviews 227:32-43 2009; Barton and Kagan, Nature Reviews Immunol 9:535-542 2009). Therefore, ablation of Unc93b1 permits efficient evaluation of the collective contributions of endolysosomal TLR signaling, by both canonical and non-canonical pathways, to MOG peptide myelitis-associated CST axonal loss.

C57BL/6 mice homozygous for an inactivating Unc93b1 missense mutation (H412R) were obtained from Mutagenetix (Tabeta et al, Nature Immunol 7:156-164 2006). MOG peptide myelitis is induced in 4 groups of EmxCre/Thy1-STOP-EYFP bone marrow chimeric mice (Cui et al, Bone Marrow Transplantation 30:843-849 2002; Remington et al, Am J Path 170:1713-1724 2007): a) wild-type Unc93b1 in both periphery and CNS; b) mutant Unc93b1 in both periphery and CNS; c) wild-type in periphery and mutant Unc93b1 in CNS; and d) mutant Unc93b1 in periphery and wild-type in CNS. Each group includes 8 mice (to ensure that at least 6 are available throughout the study), and at least 2 independent experiments are performed. The chimeric mice are examined daily, and sacrificed at day 105 post-MOG for histological evaluation, including CST axon counts as shown in FIG. 3 (see above).

### Example 4

### Deletion of MyD88 From Neuroectoderm-Derived Cells Prevents Delayed Onset, Progressive Post-Myelitic CST Axon Loss

Prior studies demonstrated that ablation of canonical TLR signaling in CNS diminishes the clinical severity of acute myelitis, though not significantly reducing the severity of acute focal axonopathy (Prinz et al, J Clin Invest 116:456-464 2006; van Loo et al, Nature Immunol 7:954-961 2006). However, the instant study establishes that deletion of MyD88 from neuroectoderm-derived cells, thus blocking canonical TLR signaling, prevents delayed onset, progressive post-myelitic CST axon loss.

A prior irradiation bone marrow chimera study (Prinz et al, 2006) showed that clinical severity of acute MOG peptide myelitis was diminished in mice that lacked expression in CNS radiation resistant cells of MyD88, a canonical TLR signaling protein, or of TLR9, an endolysosomal TLR that employs MyD88 signaling. The observation that nestinCre-mediated conditional ablation of NEMO or IKK2, proteins also instrumental in canonical TLR signaling, diminished clinical severity of MOG peptide myelitis (van Loo et al, Nature Immunol 7:954-961 2006) reinforces the importance of TLR signaling in these neuroectoderm-derived cells. Therefore constitutive nestinCre (Tronehe et al, Nature Genet. 23:99-103 1999) is used to conditionally inactivate floxed MyD88 alleles in progenitors to CNS neurons, astroglia, and oligodendroglia. Prior studies with several recombination indicator strains demonstrated that nestinCre drives greater than 90% recombination in these CNS cells (Dubois et al, Genesis 44:355-360 2006).

The first experiments are directed to nestinCre/MyD88flox/MyD88flox mice immunized with MOG peptide. Controls include nestinCre/MyD88flox/MyD88flox mice given CFA without MOG peptide, and nestinCre+ but MyD88 wild-type mice immunized with MUG peptide. There are 8 mice in each group, to ensure at least 6 mice at each time-point. Two independent experiments are performed. The mice are examined daily, and sacrificed at day 105 post-immunization. Dorsal CST axon numbers in L5 lumbar and C2 cervical spinal cord cross-sections are estimated using SMI312 immunostaining (see FIG. 4; note that intrinsic CST EYFP labeling cannot be used with this experimental design, because nestinCre would recombine Thy1-STOP-EYFP in many neuronal and macroglial populations). The sections are co-immunostained for Ibal and GFAP to evaluate microglia/macrophages and astroglia, respectively, in the dorsal CSTs. Non-biased stereology is used to count cortical layer 5 CTIP2+ corticospinal projection neurons.

### Example 5

### Inhibition of TLR Signaling By Administration of Mianserin Results in Antagonism of Axonal Degeneration

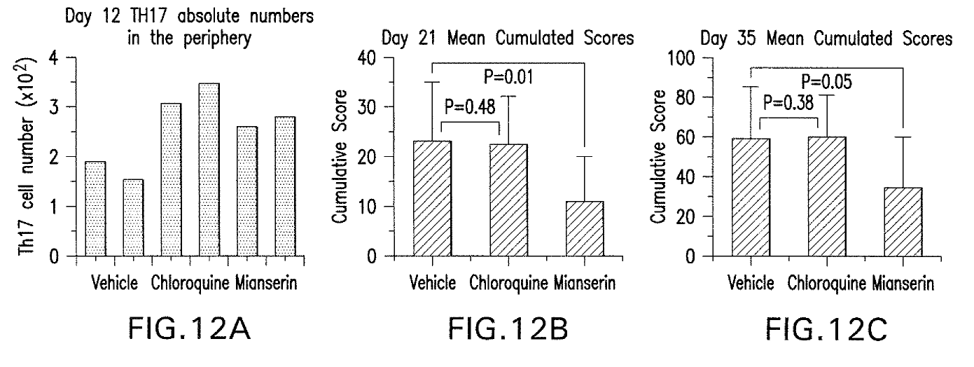

Mianserin treatment. Mice are induced with MOG-myelitis as described. Groups of mice initially receive intraperitoneally 1-10 mg/kg/day. The start day can vary between days 5-12 (before disease onset) and 14-20 (after disease onset). Peripheral T cell responses and CNS infiltration are analyzed by flow cytometry. Lumbar spinal cords are examined immunohistologically to characterize the degrees of axonal degeneration, secondary demyelination and CNS infiltration by peripheral immune cells during the various stages of MaG-myelitis. The mice are perfused with 4% paraformaldehyde and sections from lumbar spinal cord arestained with various antibodies and analyzed by confocal microscopy. In every step the degree of inflammation, axonal degeneration and demyelination is confirmed by using the following antibodies: CD11b (macrophages), CD3 (T cells), CD11c (dendritic cells), Glial fibrillary acidic protein (GFAP astroglia), Myelin Basic protein (MBP, for myelin), and SMI32 (hypophosphorylated NF—H, disrupted axons). To examine intracellular TLR and partner molecules antibodies against TLR3, 8 and 9, MyD88 and IRAK4, NALP1, ASC, active caspase-1, and IL-1 r3 are used. The resulting data is presented in FIG. 12.

Spinal cord assessment. Injured spinal cord areas are identified by either SMI32 or TLR8 immunohistochemistry. Demyelinating areas are visualized by MBP or luxol fast blue staining. Longitudinal frozen spinal cord sections are used in each case. Immunolabeling is performed using a rapid-staining method that uses gold-conjugated secondary antibodies and silver enhancement as a detection system. This system is preferred as it does not induce any protein modifications enzymatic step that could potentially induce modification on antigenic epitopes. All stains contain complete protease inhibitor cocktail (Roche). Gold particles are visualized by silver enhancement (Canemco) according to the manufacturer's instructions.

SMI32 or TLR8 rich or MBP or luxol fast blue low intensity areas of the white matter are microdissected along with unaffected areas (no axonal immunostaining for SMI32 and TLR8, or normal MBP or luxol fast blue staining of the white matter) and protein extracts are isolated and examined by western. To isolate protein extracts from microdissected areas, tissues are lysed in a urea/thiourea buffer 7M urea, 2M thiourea, 4% w/v CHAPS, 1% w/v dithiothreitol, 1 mg/ml Pefabloc) for 30 minutes at room temperature. After vigorous mixing, the samples are centrifuged at 42000 g. The supernatant is collected, assayed for protein concentration and analyzed by Western blot.

Peripheral T cell responses. Mixed splenocytes and lymph node cells are cultured in 200 μl of complete RPMI 1640 (10% FBS, 2 mM L-glutamine, 0.1 mM nonessential amino acids, 100 U penicillin-streptomycin, 50 μM 2-mercaptoethanol, and 1 mM sodium pyruvate) with or without 50 μg/mlMOG peptide (amino acids 35-55) for 24 hrs. The cells are incubated with brefeldin-A (GolgiPlug, BD Bioscience) or brefeldin A plus ionomycin (Calbiochem, 750 ng/ml) and phorbol 12-myristate 13-acetate (PMA; 50 ng/ml, Sigma-Aldrich) for the last 5 hr (Park et al, 2005).

Flow cytometry. Mixed splenocytes and lymph node cells are immunostained after culture for 24 hours as described above. CNS mononuclear cells are immunostained after incubation at 37° C. for 3 hours in complete RPMI 1640 in the presence of brefeldin A. Immediately prior to immunostaining, Fe receptors are blocked for 10 min with anti-CD16/32. For Th1/Th17 lymphocyte analysis, cells are stained with Pacific Blue-labeled anti-mouse CD4, fixed, and permeabilized using a Cytofix/Cytoperm Plus Kit according to the manufacturer's protocol, and stained with allophycocyanin (APC) labeled anti-mouse IFN-γ and phycoerythrin-labeled antimouse IL-17 (all reagents from BD Bioscience). Immunostained cells are analyzed using a Cyan FACS (Dako Cytomation).

### Example 6

### Inhibition of TLR Signaling In Genetically Modified Mice Resulting In Antagonism Of Axonal Degeneration

To inhibit intracellular TLRs functions genetically, one can interfere with their (a) location, and (b) signaling molecules. This allows for validation of the results obtained by mianserin and to further understand the role of intracellular TLRs in axonal degeneration. Two strains of genetically manipulated mice are used in this study: the ENU induced UNC93B mutant and the VAChT-Cre strains.

UNC93B chaperones intracellular TLRs (3, 7, 8 and 9) from the endoplasmic reticulum to the lysosomal compartment. Localization of intracellular TLRs to the lysosomes is critical for their function. Both human and mice with inactive UNC93B gene lack endosomal TLR responses. Bone marrow chimeras are created in which mice with a missense mutation is lethally irradiated and receives bone marrow cells from control mice. In the resulting chimeras UNC93B activity is abolished only in the CNS. Peripheral immune cells carry the active form of UNC93B, so the generation, activation, polarization and migration of pathogenic T cells is not be affected. The degree of axonal degeneration and clinical symptoms observed in MOG-myelitis induced mice carrying the non-active form of UNC93B, in the CNS, is lower than their control counterparts. C57BL/6 mice, carrying the CD45.2 allele, are lethally irradiated (900 cGy), and 20 hrs later they receive bone marrow cells isolated from a congenic strain that carries the CD45.1 allele (C57BL/6-Ly5.1). CD45 is expressed on all nucleated cells and the use of these allelic variants makes it convenient to follow the transplanted bone marrow cells. Splenocytes isolated from C57BL16Ly5.1 mice are only CD45.1+ while splenocytes isolated from C57BL/6 carry only the CD45.2 allele. After iv injection of bone marrow cell from C57BL/6-Ly5.1 (donor) to irradiated C57BL/6 mice (recipient), the resulting chimeras contain 94.2% of CD45.1+ splenocytes while microglia maintained the CD45.2 allele. The fact that peripheral cells contain 5.4% of CD45.2+ cells is probably due to the low irradiation dosage. The mice are subjected to the lowest dosage of irradiation documented. However, CS7BL/6 mice can endure up to 1200 cGy.

The second strain that is employed to examine the contribution of intracellular TLR signaling on axonal degeneration has a deletion of MyD88 specifically in adult motor neurons. Mice lacking MyD88 in neurons are generated by crossing floxed MyD88 to Vesicular AcetylCholine Transporter (VAChT)-Cre. To follow recombination efficiency, these mice are also crossed to the ROSA-EYFP reporter mouse. Immunohistological labeling with ChAT in parallel with axonal degeneration markers allows for the determination of any difference in the degree of degeneration between axons that are MyD88-deficient (EYFP+) or MyD88-sufficient (EYFP−). The double transgenic strain: VAChT-Cre/+ROSASTOP-EYFP+/+ serve as control mice when clinical symptom severity is compared with the triple transgenics.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. Furthermore, patents, patent applications, publications, procedures, and the like are cited throughout this application, the disclosures of those materials are hereby expressly incorporated herein by reference in their entireties.

What is claimed is:

1. A method for inhibiting axonal degeneration comprising inhibiting the activity of a Toll-Like Receptor to thereby inhibiting axonal degeneration.

2. The method of claim 1 wherein inhibition of the activity of a toll like receptor is induced by administration of a Toll-Like Receptor inhibitor selected from the group consisting of small molecule Toll-Like Receptor inhibitors and biologic Toll-Like Receptor inhibitors.

3. The method of claim 2 wherein the small molecule Toll-Like Receptor inhibitor is selected from the group consisting of tricyclic Toll-Like Receptor inhibitors, substituted quinoline compounds and substituted quinazoline compounds.

4. The method of claim 3 wherein the tricyclic Toll-Like Receptor inhibitor is selected from the group consisting of mianserin, desipramine, cyclobenzaprine, imiprimine, ketotifen, and amitriptyline.

5. The method of claim 2 wherein the biologic Toll-Like Receptor inhibitor is selected from the group consisting of Toll-Like Receptor inhibiting antibodies, Toll-Like Receptor inhibiting peptides, and Toll-Like Receptor inhibiting nucleic acids.

6. A method for treating axonal loss associated with myelitis comprising inhibiting the activity of a Toll-Like Receptor to inhibit axonal degeneration, thereby treating axonal loss associated with myelitis.

7. A method for treating axonal loss associated with multiple sclerosis comprising inhibiting the activity of a Toll-Like Receptor to inhibit axonal degeneration, thereby treating multiple sclerosis.

8. A method for treating axonal loss associated with systemic lupus erythematosis comprising inhibiting the activity of a Toll-Like Receptor to inhibit axonal degeneration, thereby treating axonal loss associated with systemic lupus erythematosis.

9. A method for treating axonal loss associated with acute corticospinal tract trauma comprising inhibiting the activity of a Toll-Like Receptor to inhibit axonal degeneration, thereby treating axonal loss associated with acute corticospinal tract trauma.

10. A method for treating axonal loss associated with viral infection comprising inhibiting the activity of a Toll-Like Receptor to inhibit axonal degeneration, thereby treating axonal loss associated with viral infection.
